(19) United States
(12) Patent Application Publication
(10) Pub. No.: US 2007/0038115 A1
(43) Pub. Date: Feb. 15, 2007

(54) High intensity ultrasound apparatus methods and systems

(76) Inventors: David P. Quigley, (US); Drew J. Tomasik, (US)

Correspondence Address: Intellectual Property Group;Bose McKinney & Evans LLP, 2700 First Indiana Plaza, 135 North Pennsylvania Street, Indianapolis, IN, 46204, US

(21) Appl. No.: 11/337,626

(22) Filed: Jan. 23, 2006

Related U.S. Application Data
(60) Provisional application No. 60/707,914, filed on Aug. 12, 2005.

(51) Int. Cl. A61B 8/14

(52) U.S. Cl. 600/471

(57) ABSTRACT

A surgical clamp including a first arm and a second arm, a source of high intensity unfocused ultrasound the source coupled to the first arm, and an ultrasound reflector coupled to the second arm is provided. At least one of the first arm and the second arm is moveable to position the reflector to reflect non-focused high intensity ultrasound emitted by the source and to clamp an organ or tissue intermediate the source and the reflector.

### CROSS REFERENCES

This application claims the benefit of U.S. Provisional Patent Application Ser. No. 60/707,914, entitled “High Intensity Reflected Ultrasound System,” filed on Aug. 12, 2005, and the same is expressly incorporated herein by reference.

### BACKGROUND

The present invention relates to surgical apparatus, systems and methods including or utilizing a surgical clamp having an ultrasound source.

Ultrasound techniques have contributed greatly to the advancement of medical treatment and diagnosis. Ultrasound has been useful in diagnosing and treating various disorders, illnesses, conditions and diseases. Presently there is a need to deliver diagnostic and therapeutic ultrasound in surgical applications in connection with or in lieu of other care. Various embodiments of the present invention address this and other needs.

### SUMMARY

In general, embodiments of the present invention provide devices, systems and methods relating to high intensity ultrasound surgical clamps. One embodiment of the present invention provides a surgical clamp including a first arm and a second arm, a source of high intensity unfocused ultrasound coupled to the first arm, and an ultrasound reflector coupled to the second arm. In this embodiment at least one of the first arm and the second arm is moveable to position the reflector to reflect non-focused high intensity ultrasound emitted by the source and to clamp an organ or tissue intermediated the source and the reflector.

Another embodiment of the present invention provides a system including a surgical clamp having a multi-mode ultrasound transducer and a controller operatively coupled to the transducer to power the transducer. In this embodiment the controller is operable in a first mode to power the transducer to emit therapeutic ultrasound and is further operable in a second mode to power the transducer to emit diagnostic ultrasound, including feedback, imaging, and/or non-imaging ultrasound.

A further embodiment of the present invention provides a method including providing a medical instrument including a clamp and an ultrasound transducer coupled to the clamp, clamping tissue with the clamp, subjecting clamped tissue to high intensity ultrasound emitted by the transducer, determining a characteristic of the clamped tissue using ultrasound emitted by the transducer.

Additional aspects and advantages of the present invention will be apparent from the following description.

### BRIEF DESCRIPTION OF THE DRAWING

FIG. 1 is a simplified block diagram of a surgical system according to one embodiment of the present invention;

FIG. 2 is a perspective view of a controller/monitor according to one embodiment of the present invention;

FIG. 3 is a perspective view of surgical device according to one embodiment of the present invention;

FIG. 4 is a sectional view of a portion of a surgical device according to one embodiment of the present invention;

FIG. 5 is a perspective view of an articulating arm and a surgical device according to one embodiment of the present invention;

FIG. 6 is a side view of a portion of a surgical device according to one embodiment of the present invention;

FIG. 7 is a side view of a portion of a surgical device according to one embodiment of the present invention;

FIG. 8 is an internal view of a portion of a surgical device according to one embodiment of the present invention;

FIG. 9 is a sectional view of a portion of a surgical device according to one embodiment of the present invention;

FIG. 10 is an internal view of a portion of a surgical device according to one embodiment of the present invention;

FIG. 11 is an internal view of a portion of a surgical device according to one embodiment of the present invention;

FIG. 12 is a perspective view of an organ;

FIG. 13 is a perspective view of a surgical device according to one embodiment of the present invention and an organ;

FIG. 14 is a sectional view of a surgical device according to one embodiment of the present invention and an organ;

FIG. 15 is a perspective view of a surgical device according to one embodiment of the present invention and an organ;

FIG. 16 is a sectional view of a surgical device according to one embodiment of the present invention and an organ;

FIG. 17 is a perspective view of an organ;

FIG. 18 is a perspective view of a surgical device according to one embodiment of the present invention and an organ; and

FIG. 19 is a side view of a surgical device according to one embodiment of the present invention and an organ.

### DETAILED DESCRIPTION

For the purposes of promoting an understanding of the principles of the invention, reference will now be made to the embodiments illustrated in the drawings and specific language will be used to describe the same. It will nevertheless be understood that no limitation of the scope of the invention is thereby intended, such alterations and further modifications in the illustrated device, and such further applications of the principles of the invention as illustrated therein being contemplated as would normally occur to one skilled in the art to which the invention relates.

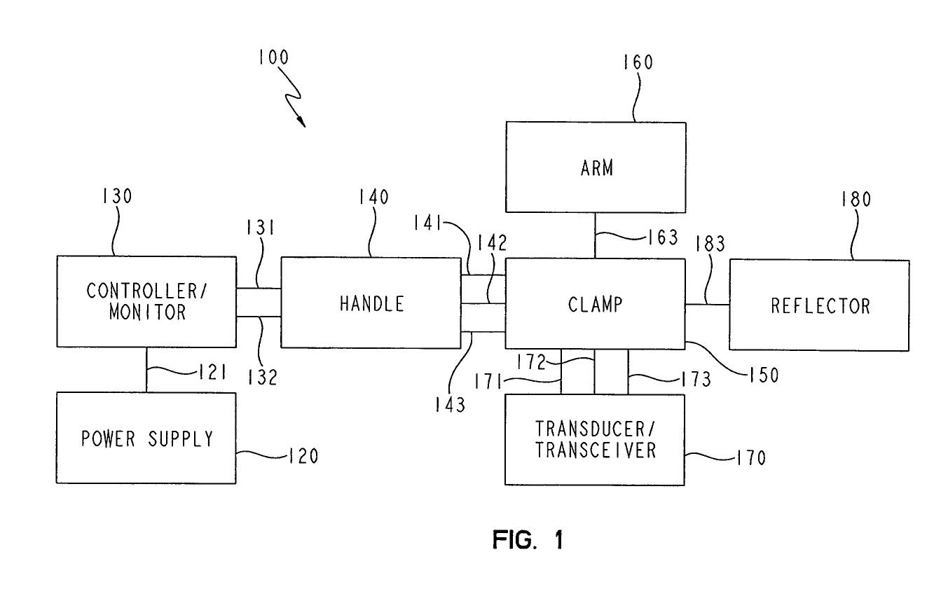

With reference to FIG. 1 there is shown a block diagram of a system 100 according to one embodiment of the present invention. System 100 includes controller/monitor 130 which is electrically coupled to and powered by power supply 120 via power coupling 121. The controller/monitor 130 supplies power to a clamp handle 140 via power coupling 131, which supplies power to a clamp 150 via power coupling 141, which supplies power to ultrasound transducer or transceiver 170 via power coupling 171. Power couplings 121, 131, 141, and 171 may include, for example, one or more insulated electrically conductive wires or power cords and may also include one or more junctions, connectors, splitters, transformers, rectifiers, fuses, and/or switches for distributing and controlling the distribution of power to various elements of system 100 and allowing releasable interconnection of the various elements of system 100.

System 100 also allows the transmission of information, such as electrical signals between various components of system 100. Information coupling 132 couples controller/monitor 130 and handle 140, information coupling 142 couples handle 140 and clamp 150, and information coupling 172 couples clamp 150 and transducer or transceiver 170. Information couplings 132, 142 and 172 may include, for example, one or more electrically conductive transmission wires, printed circuit boards including electrically conductive pathways and/or electrical connectors and could also include fiber optics, transmitters and receivers or other couplings for transmitting information. Information couplings 132, 142 and 172 allow transmission of information to, from and between various components of system 100. In one embodiment, it is contemplated that information couplings 132, 142 and 172 constitute a single transmission route that couples information between controller/monitor 130 and transducer or transceiver 170.

System 100 further includes mechanical interconnections or couplings. Handle 140 and clamp 150 may be mechanically coupled and decoupled at mechanical coupling 143. Similarly, information coupling 142 and power coupling 141 may be selectably coupled and decoupled to permit clamp 150 and handle 140 to be joined and separated as desired. Clamp 150 may be mechanically coupled to transceiver or transducer 170 at coupling 173, to reflector 180 at coupling 183 and coupled and decoupled to articulating arm 160 at mechanical coupling 163. It is contemplated that couplings 143, 163, 173, and 183 could include any couplings or interconnections including those described below in further detail. Additional exemplary attributes of the components of system 100 are also described below.

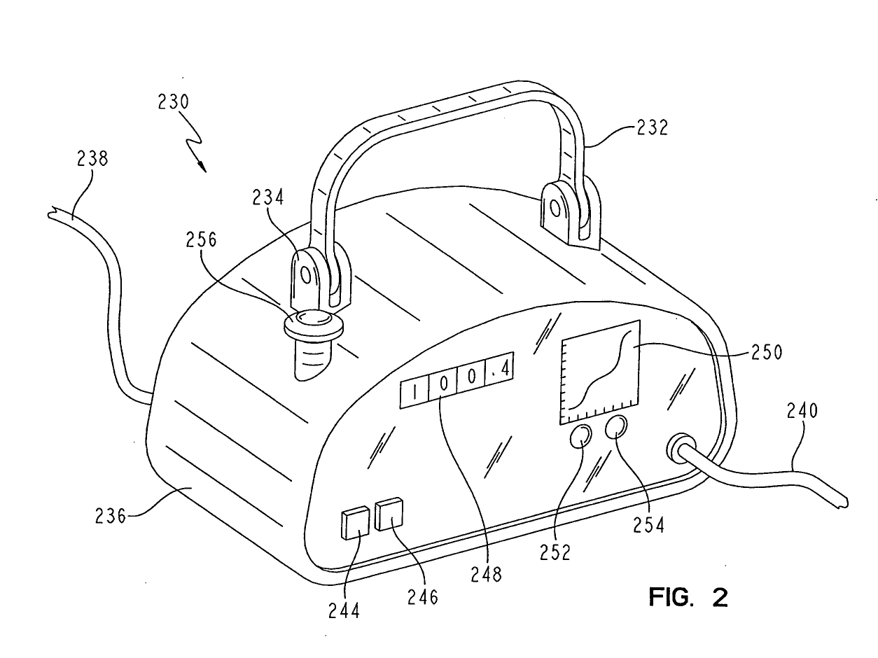

With reference to FIG. 2 there is shown a monitor/controller 230 according to one embodiment of the present invention. Monitor/controller 230 is illustrated as a portable unit housed in cabinet 236 and including a handle 232 connected to cabinet 236 at hinge 234. It is also contemplated that monitor/controller 230 could be a dedicated unit or could be housed on a cart to allow positioning or transportation. Cabinet 236 and handle 232 are made of water resistant, non-reactive materials that permit easy cleaning such as stainless steel, hospital grade plastics or other such materials.

Monitor/controller 230 is connected to and powered by a power source, such as an electrical outlet, via a detachable hospital grade power cord 238. Monitor/controller 230 outputs electrical power suitable for driving an ultrasound transducer or transceiver via power output cord 240 and includes an emergency stop switch 256 for interrupting power output. Cord 240 may also include a separate information pathway such as one or more wires for transmitting and/or receiving information. An information pathway may also be provided through a separate cord, wire, or wire bundle or by various other information couplings, such as those previously described.

Monitor/controller 230 includes on and off switches 244 and 246, which may optionally be a single switch, digital display 248, monitor 250, controls 252 and 254 and may include additional controls such as an alphanumeric keypad or a computer interface for receiving control or diagnostic input from a computer and sending information to a computer or computing device. Display 248 permits the display of digital alpha-numeric information. For example, display 248 can be used to display temperature of a remotely located ultrasound transducer, thermistor, or transceiver that is calculated by a microprocessor of monitor/controller 230 based upon an electrical signal received from a sensor located at or near the remote transducer or transceiver. Display 248 may be a LED display that is electrically coupled to and driven by a microprocessor or other driver circuitry.

Monitor 250 may be a CRT monitor, LCD display, plasma display or other display suitable for depicting graphical information. In one example, display 250 displays a graph that depicts the rate of blood flow through an organ or tissue as a function of time. The display is generated based upon information received from a remote ultrasound transducer employing a Doppler ultrasound technique. The rate of blood flow of a tissue or organ can be determined using a microprocessor or other circuitry based on information received from the pickup or receiver of an ultrasound transducer or transceiver and transmitted to monitor/controller 230 via one or more information couplings. In another example, display 250 displays graphical information of the degree of ablation or cauterization of a tissue or organ based upon information received from an ultrasound transducer using an elastography ultrasound technique. The degree of ablation or cauterization can be determined using microprocessor techniques based upon information received from the pickup or receiver of an ultrasound transceiver corresponding to the elasticity of the tissue or organ and transmitted to monitor/controller 230 via one or more information couplings. In still another example monitor 250 can display information indicating the mode of operation the monitor/controller 230, for example whether monitor/controller 230 is outputting power and/or control signals appropriate for a remote ultrasound transducer to operate in a Doppler ultrasound mode, an elastography ultrasound mode or a high intensity ultrasound mode. Alternatively, this information can be displayed on display 248.

Controls 252 and 254 and/or similar controls can be used to change the information displayed on monitor 250 and to adjust the operation of monitor/controller 230, for example to cause monitor/controller 230 to output power and/or control signals appropriate for a remote ultrasound transducer to operate in a Doppler ultrasound mode, in an elastography ultrasound mode or a high intensity ultrasound mode. These or similar controls could also be used to dynamically adjust power levels of high intensity ultrasound in a therapeutic mode of operation.

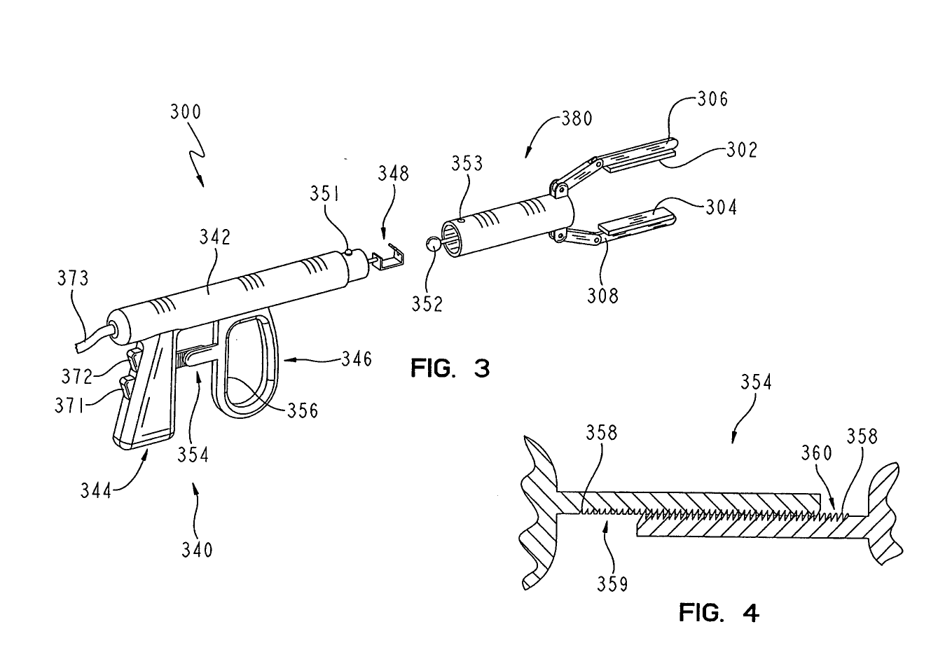

With reference to FIG. 3 there is shown surgical instrumentation 300 according to one embodiment of the present invention. Instrumentation 300 includes handle 340 and clamp 380. Handle 340 includes tubular housing 342 which is formed of stainless steel or other material which can be readily sterilized as may be required for a surgical environment. Handle 340 further includes hand grips 344 and 346 which are formed of surgical grade plastic or other material which can be readily sterilized as may be required for a surgical environment. Other materials suitable for a surgical environment can also be used to construct handle 340 and the other components of instrumentation 300.

Handle 340 is connected to a controller/monitor, for example monitor/controller 230 illustrated in FIG. 2, via cord 373 which provides for transmission of power suitable to drive an ultrasound transducer to instrumentation 300. Cord 373 may also provide for transmission of information to or from instrumentation 300 to a remotely located controller/monitor via one or more transmission wires or other information couplings. Alternatively, information couplings may be independent from cord 373, such as through a separate cord.

Handle 340 includes operator control buttons 372 and 371 which are operable by the user to control the operation of an ultrasound transducer. In one embodiment button 372 is a safety that must be released to permit operation of the transducer and button 371 is a trigger that operates the transducer when the safety is released. Handle 340 also includes grip locking mechanism 354 and finger handle 356 which can control the actuation of clamp 380.

Clamp 380 includes clamp arms 306 and 308 which may be actuated as described below. Ultrasound transducer 302 is coupled to and moves with clamp arm 306. Ultrasound transducer 302 may be a flat ultrasound transducer and may be ultrasonically insulated on the surface that is attached to clamp arm 306, for example, with an air pocket or other ultrasonic insulator, and when energized emits ultrasound on its opposite face. Ultrasound transducer 302 may also be an array of two or more ultrasound transducers, such as an array of hinged ultrasound transducers arranged, for example, in a line or other configuration. Ultrasound transducer 302 is configured to transmit and receive a variety of types of ultrasound. For example, transducer 302 may transmit and receive ultrasound appropriate for Doppler ultrasound techniques, elastography ultrasound techniques, and high intensity ultrasound techniques. Transducer 302 may be configured to perform particular high intensity ultrasound techniques such as high intensity focused ultrasound or high intensity unfocused ultrasound. In combination with ultrasound reflector 304, transducer 302 may provide high intensity reflected ultrasound. Ultrasound transducer 302 may have the capacity of operating to emit one or more of the foregoing types of ultrasound, and in the case of Doppler and elastography ultrasound to receive signals through one or more pickups, receivers or detectors.

Ultrasound transducer 302 may be encapsulated in a leak proof membrane which is transparent to ultrasound to permit transmission of ultrasound to a treatment site. Transducer 302 may also include an ultrasound coupling element, such as degassed water which is contained within the leak proof membrane and facilitates the propagation of ultrasound. The degassed water also cools the transducer and may be circulated using a pump to increase cooling efficacy. Transducer 302 may also directly contact an organ or tissue to create a water-based tissue interface or sterilized ultrasound gel may be placed on the transducer surface to create an ultrasonic interface.

The ultrasound transducer 302 is connected to a power supply via wires which are routed internally through clamp 380 and handle 340 or through other power couplings to a controller/monitor, such as monitor/controller 230 illustrated in FIG. 2. In this manner electrical signals can be applied to one or more piezoelectric elements of transducer 302 to cause it to emit ultrasound. Ultrasound transducer 302 can be driven in a variety of manners, depending on the nature of the desired ultrasound emission. In one mode of operation, the transducer is powered by electrical signals in the range of about 1 MHz to 30 MHz to transmit high intensity ultrasound having the capacity to cauterize tissue. In another mode of operation the, transducer is powered by electrical pulse signals appropriate to produce diagnostic ultrasound where a receiver portion of the transducer receives reflected ultrasound energy which it converts to an electrical signal that is sent via information transmission wires routed internal to clamp 380 and handle 340 or through other information couplings to a controller/monitor.

Various types of diagnostic ultrasound operation are contemplated, including feedback, imaging, and/or non-imaging ultrasound. For example, Doppler ultrasound can be used to determine a rate of blood flow in a tissue and elastography ultrasound can be used to determine the elastic properties of a tissue. A temperature thermistor or other temperature sensor can also be attached to, near or within ultrasound transducer 302. Additionally, a pressure sensor can be attached to, near or within ultrasound transducer 302. Information from transducer 302 and any associated temperature or pressure sensor can be transmitted through one or more wires or other information transmission pathways routed internally through clamp 380 and coupled to wires or other information transmission pathways of handle 340 which are in turn coupled to a controller/monitor. Thus, in conjunction with a controller/monitor ultrasound transducer 302 may have the capacity for cauterizing tissue, determining a rate of blood flow in tissue, determining tissue elasticity, determining temperature associated with transducer 302 and/or determining pressure associated with transducer 302.

Ultrasound reflector 304 is coupled to clamp arm 308. Reflector 304 is polished stainless steel that reflects high intensity ultrasound energy emitted by transducer 302 when operating a high intensity ultrasound mode to cauterize tissue. Reflector 304 could also be any other material which absorbs or reflects ultrasound. During operation of transducer, reflector 304 can define a boundary beyond which ultrasound emitted by transducer 302 does not pass.

As illustrated in FIG. 3, clamp 380 includes clamp connector 352 and handle 340 includes handle connector 348. Handle connector 348 can receive and retain clamp connector 352 to form a mechanical interconnection between handle 340 and clamp 380 permitting the transfer of mechanical force, and can also release handle connector 352. Additionally, connector 352 and 348 can establish electrical interconnections between handle 340 and clamp 380 through one or more pairs of electrical contacts and associated internally routed wires. An electrical interconnection can also be provided through separate internally routed wires. The electrical interconnections between clamp 380 and handle 340 can be used to drive transducer 302 and send and receive information.

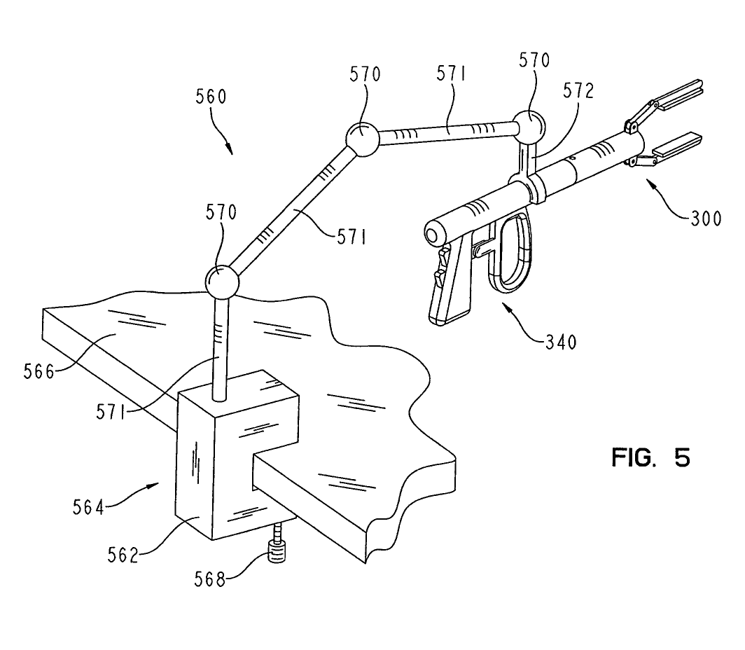

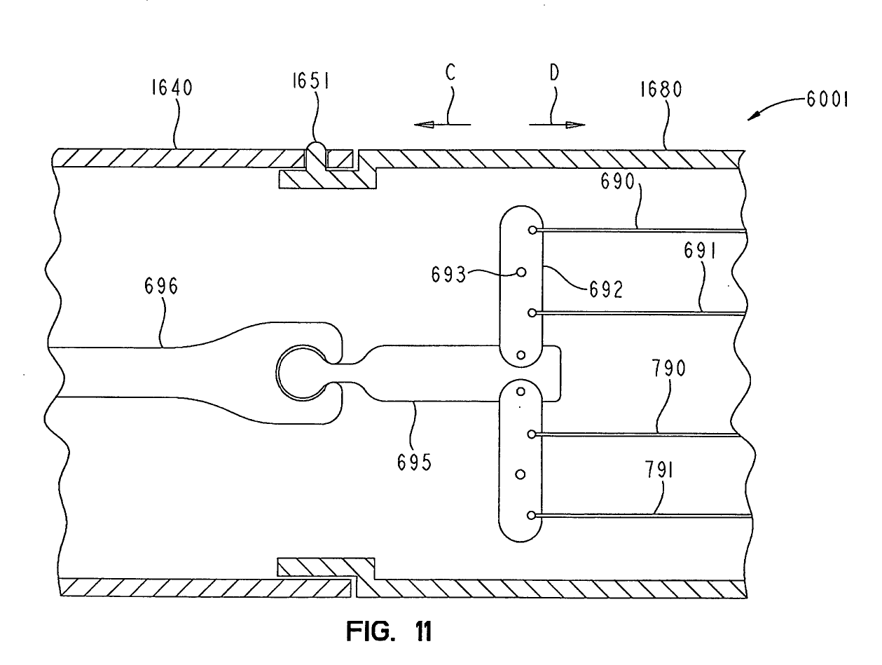

Clamp 380 and handle 340 are configured to be interconnected by the end of handle 340 being inserted into the end of clamp 380, and locking mechanism 352 releasably engaging with receptacle 353. This interconnection is illustrated in FIGS. 5 and 11. It is also contemplated that a variety of other interconnections could be established between clamp 380 and handle 340, for example, a twist lock mechanism could be provided at the end of handle 380 to rotatably engage one or more studs on clamp 340, a threaded collar of handle 340 could attach to threads on the exterior of clamp 340, and still other interconnections could be used. Regardless of the type of interconnection used, it is contemplated that clamp 380 could be a disposable or single use device, while handle 340 could be reusable. Alternatively, clamp 380 could be a multiple use device that could also be sterilized and reused. It is also contemplated that handle 340 and clamp 380 could be a single piece instrument.

With reference to FIG. 4 there is shown a more detailed illustration of grip locking mechanism 354. Grip locking mechanism 354 includes serrated surfaces 359 and 360 and maximum stop points 358. Grip locking mechanism provides controlled indexed movement of hand grips 344 and 346 relative to one another. When such force is applied to finger handle 356 in the direction toward hand grip 344 the serrations of serrated surfaces 359 and 360 incrementally engage and release to accommodate movement in this direction. When such force is no longer applied, serrated surfaces 359 and 360 engage to maintain the current position. Movement in the opposite direction is similarly accommodated.

With reference to FIG. 5 there is shown an articulated instrument holder 560 which includes a number of articulating joints 570 and arm sections 571. Instrument holder 560 includes a base 564 that is secured to surgical table 566 with knob 568 which can be tightened and loosened to permit installation, removal and positioning of instrument holder 560. Base 564 may also include an adaptor (not shown) for attaching to other articulated arms as may be required in adjunctive therapy situations.

Instrument holder 560 further includes a coupler 572 which couples holder 560 to instrumentation 300. As illustrated coupler 572 is a clamp that attaches to handle 340 of instrumentation 300. It is contemplated, however, that a variety of couplings could be employed. Joints 570 are multiaxial and may be adjusted to articulate at any angle to maintain instrumentation 100 in a desired position.

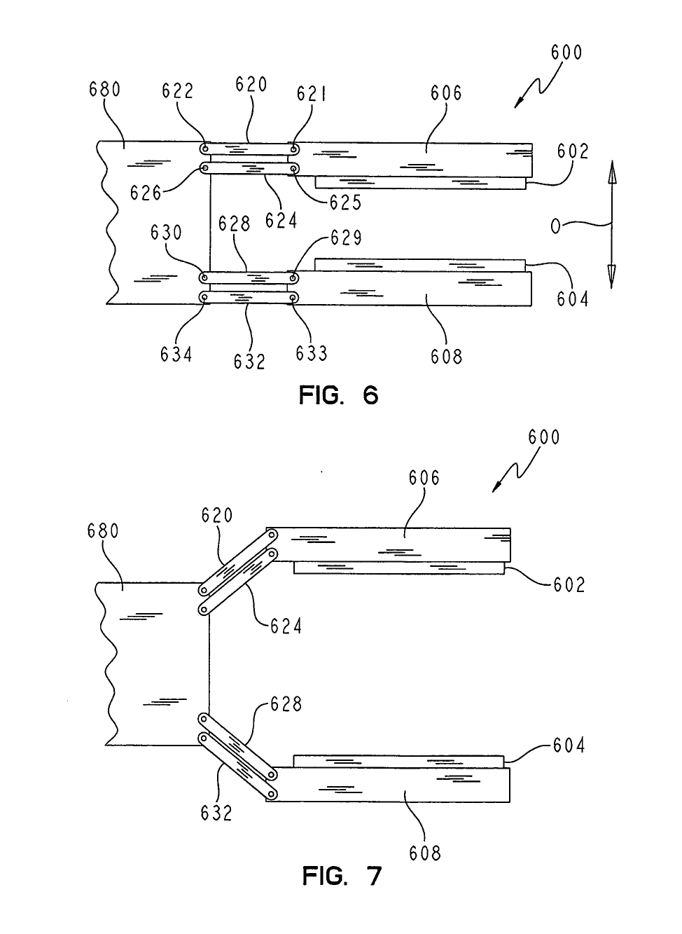

With reference to FIG. 6 there is shown a portion of a surgical clamp 600 according to one embodiment of the present invention. As illustrated in FIG. 6, clamp 600 includes clamp arms 606 and 608, clamp body 680, and clamp linkages 620, 624, 628 and 632 which interconnect clamp arms 606 and 608 and clamp body 680. Clamp 600 further includes ultrasound transducer 602 and ultrasound reflector 604 which may be the same or similar to the ultrasound transducers and reflectors described herein. Clamp linkages 620 and 624 are rotatably connected to clamp arm 606 and clamp body 680 with axles or shafts 621, 622, 625, and 626. Clamp linkages 628 and 632 are rotatably connected to clamp arm 608 and clamp body 680 with axles or shafts 629, 630, 633, and 634.

The interconnection of clamp arms 606 and 608 and clamp body 680 permits clamp arms 606 and 608 to move generally in the direction indicated by arrow O in order to move clamp arms 606 and 608 apart and also to move in the opposite direction in order to move clamp arms 606 and 608 together. Linkages 620 and 624 form a parallelogram linkage between clamp arm 606 and clamp body 680 which maintains the angular relationship between clamp arm 606 and clamp body 680 during movement of clamp arm 606. Similarly, linkages 628 and 632 form a parallelogram linkage between clamp arm 608 and clamp body 680 which maintains the angular relationship between clamp arm 608 and clamp body 680 during movement of clamp arm 608. The angular relationship between clamp arm 606 and clamp arm 608 is also maintained during movement of the clamp arms 606 and 608.

With additional reference to FIG. 7 there is shown surgical clamp 600 as was just described in connection with FIG. 6. In FIG. 6 clamp 600 was illustrated in a configuration in which clamp arms 606 and 608 were in a relatively closed or low profile configuration. In FIG. 7 clamp 600 is illustrated in a different configuration in which arms 606 and 608 have been moved apart in the direction of arrow O illustrated in FIG. 6 to a relatively more open configuration. Clamp arms 606 and 608 can also be moved to positions intermediate those illustrated in FIG. 6 and FIG. 7, to even more open positions, and to even more closed positions.

It is contemplated that surgical clamp 600 could vary in a number of manners to facilitate greater or lesser ranges of movement of clamp arms 606 and 608. For example, the position of axles 622 and 626 could be offset with axle 622 being located farther to the left, and the position of axles 621 and 625 could be offset with axle 625 being located farther to the right to permit greater range of motion of clamp arm 606 and a similar offset could be provided between axles 630 and 634, and axles 629 and 633 to permit greater range of motion of clamp arm 608. The length of linkages 620, 624, 628, and 632 could be increased to provide greater range of motion or decreased to provide lesser range of motion. The thickness of linkages 620, 624, 628, and 632 could be increased to reduce range of motion or decreased to increase range of motion. The closeness or shape of linkages could also vary to increase or decrease range of motion. For example the linkages could be curved, bent, compound, jointed, tapered, or otherwise modified to accommodate or facilitate the particular range of motion desired. Furthermore, one or more stops or other structures could physically limit range of movement.

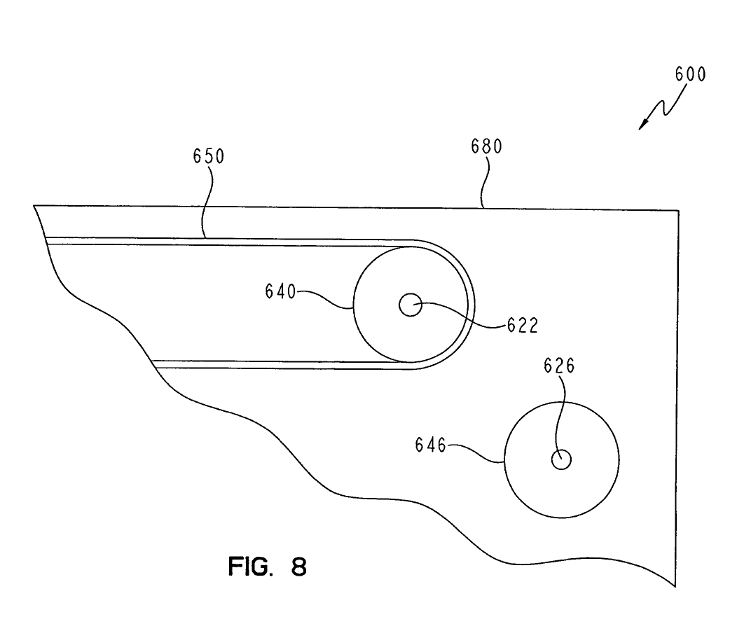

With reference to FIG. 8 there is shown a portion of a surgical clamp 600 as was just described in connection with FIGS. 6 and 7. In FIG. 8 a portion of the interior of clamp body 680 is illustrated including drive wheel 640, drive axle 622, free wheel 646, free axle 626 and drive belt 650. As described above, surgical clamp 600 can be operated so that clamp arms 606 and 608, illustrated in FIGS. 6 and 7, can be moved. This movement may be controlled by a surgeon who manipulates controls on a clamp handle such as those described below in connection with FIG. 9. Movement of the controls causes a corresponding movement of drive belt 650 which, in turn, causes drive wheel 640 to rotate in either a clockwise or counter clockwise direction. Rotation of drive wheel 640 causes rotation of drive axle 622 in the same direction. Drive axle 622 extends through clamp body 680 and is coupled to linkage 620 which was illustrated and described above in connection with FIGS. 6 and 7. Through this connection, rotation of axle 622 causes movement of linkage 620 which, in turn causes movement of clamp arm 606 as was described above in connection with FIGS. 6 and 7. Free wheel 646 is connected to and can rotate with and free axle 626 which extends through clamp body 680 and is coupled to linkage 624 which was illustrated and described above in connection with FIGS. 6 and 7. Free wheel 626, free axle 646 and linkage 624 rotate together in passive response to the movement of clamp arm 606 as was described above. Additionally, a similar drive belt, drive wheel, drive axle, free wheel, and free axle are interconnected with linkages 628 and 632 to provide for the controlled movement of clamp arm 608.

A number of variations and modifications to the features described and illustrated above in connection with FIG. 8 are contemplated. For example, free wheel 646 and free axle 626 could be driven by a second drive belt that was actuated along with drive belt 650 or wheels 640 and 646 could be interconnected with an interconnection belt so that rotation of wheel 640 would cause rotation of wheel 646. A similar interconnection belt could be used to interconnect and drive one or more wheels associated with linkages 628 and 632. It is contemplated that the drive belts and interconnection belts could be flat belts, or could have a geared inner surface and that wheels of surgical clamp 600 could also have gear teeth around their circumference to engage the geared surface of the belts. It is further contemplated that the belts of surgical clamp 600 could be chains.

With reference to FIG. 9 there is shown an internal view of a portion of a clamp handle 642. Handle 642 includes adjuster 670 which is connected to drive belt 650. Adjuster 670 can be moved in the directions indicated by arrows A and B to cause a corresponding movement of drive belt 650. As was described above in connection with FIGS. 7 and 8, the movement of drive belt 650 is used to control the movement of clamp arms 606 and 608. Adjuster 670 therefore provides user control of the movement of clamp arms 606 and 608. As was previously described in connection with FIGS. 3 and 4 adjuster 670 may slide in the direction of arrows A and B and may include a grip locking mechanism. It is also contemplated that adjuster 670 could pivot at a point 672 shown in FIG. 9 to accomplish movement of belt 650.

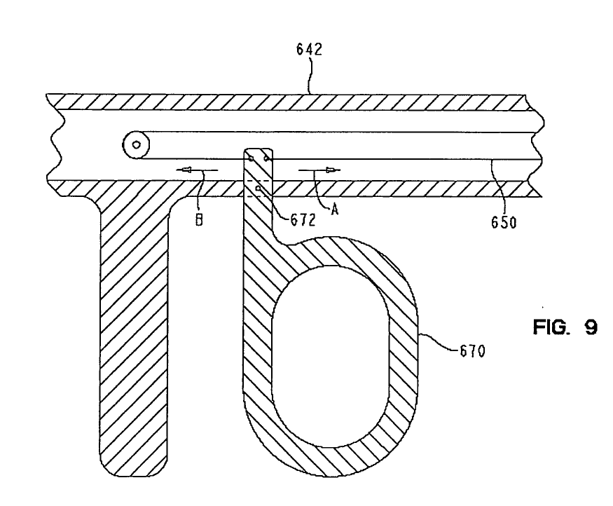

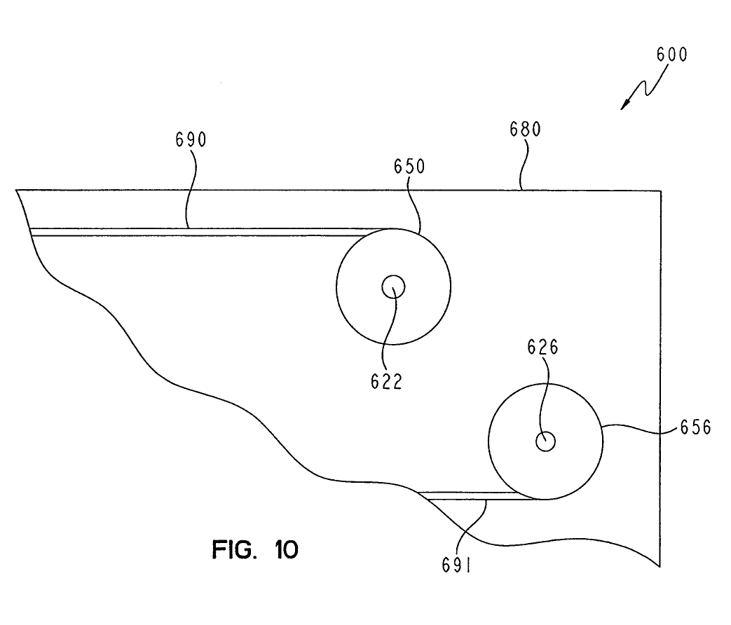

With reference to FIG. 10 there is shown an internal view of a portion of a clamp handle 642 of an alternate embodiment of surgical clamp 600. In FIG. 10 a portion of the interior of clamp body 680 is illustrated including spool 650, axle 622, wire 690, spool 656, axle 626 and wire 691. As described above, surgical clamp 600 can be operated so that clamp arms 606 and 608, illustrated in FIGS. 6 and 7, can be moved. This movement may be controlled by a surgeon who manipulates controls on a clamp handle similar to those described above in connection with FIG. 9. Movement of the controls causes wires 690 and 691 to move in opposite directions which, due to wires 690 and 691 being wound around their respective spools in opposite directions, causes spools 650 and 656 to rotate in the same direction. As wire 690 moves in a direction away from spool 650 it unwinds from spool 650 and as it moves in a direction toward spool 650 it winds around spool 650. Similarly, as wire 691 moves in a direction away from spool 651 it unwinds from spool 651 and as it moves in a direction toward spool 651 it winds around spool 651. Rotation of spools 650 and 656 causes rotation of axles 622 and 626 in the same direction. As was described above axle 622 and 626 extend through clamp body 680 and are coupled to linkages 620 and 624 which were illustrated and described above in connection with FIGS. 6 and 7. The rotation of axles 622 and 626 causes opening and closing of the clamp arms of clamp 600 as was described above in connection with FIGS. 6, 7 and 8.

With reference to FIG. 11 there is shown a cross section of a portion of surgical device 6001 according to the present invention. Surgical device 6001 includes clamp handle 1640 and clamp 1680 which are only partially illustrated. Clamp handle 1640 and clamp 1680 are separable pieces that fit together and are held in position by interlock 1651. Clamp handle 1640 includes control rod 696 which is connected to and moved by user controls such as finger handle 356 and hand grip 346 described above in connection with FIG. 3. Control rod 696 releasably interconnects with control rod 695 of clamp 1680 and is effective to transfer motion between the two control rods. Thus when control rod 696 is moved in the direction of arrow C control rod 695 is also moved in the direction of arrow C.

Control rod 695 is coupled to control lever 692 which rotates about central axle 693 which is connected to clamp 1680. Wires 690 and 691 are connected to lever 692 on opposite sides of axle 693. Wire 690 continues to a spool such as spool 650 which was illustrated and described above in connection with FIG. 10. Wire 691 continues on to a spool such as spool 656 which was also which was also illustrated and described above in connection with FIG. 10. This interconnection further translates movement of control rod 696 to wires 690 and 691. For example, when control rod 696 moves in the direction or arrow C control rod 695 also moves in that direction. Consequently, the lever 692 rotates around axle 693 in a clockwise fashion. This, in turn causes wire 690 to move in the direction of arrow D and wire 691 to move in the direction of arrow C. This causes spools 650 and 656 to rotate in a clockwise direction and, in turn, the closing movement of clamp arm 606 is accomplished as was previously described in connection with FIGS. 6-8. Similarly, when control rod 696 moves in the direction of arrow D the opposite movement is achieved and clamp arm 606 can open. A similar lever and axle are provided for wires 790 and 791 which are connected to similar spools to control movement of clamp arm 608. It is also contemplated that wires 690 and 691 could be substituted for a belt or chain and interconnected with one or more drive wheels as was described in connection with FIG. 8 above. Furthermore, it is contemplated that control rods 696 and 695 could provide electrical interconnection and was described in connection with FIG. 3.

With reference to FIG. 12 there is shown a portion of an organ 1254 which includes vasculature 1252 having bloodflow therethrough indicated by arrow 1256. Organ 1254 is illustrated generically, but could be any organ or tissue, for example, a kidney, gall bladder pancreas, spleen, uterus, stomach, intestine, blood vessel, liver, appendix or other organs of the cardiovascular system, digestive system, endocrine system, immune system, lymphatic system, muscular system, nervous, reproductive system, respiratory system, skeletal system, urinary system, or organs or tissues of other body systems.

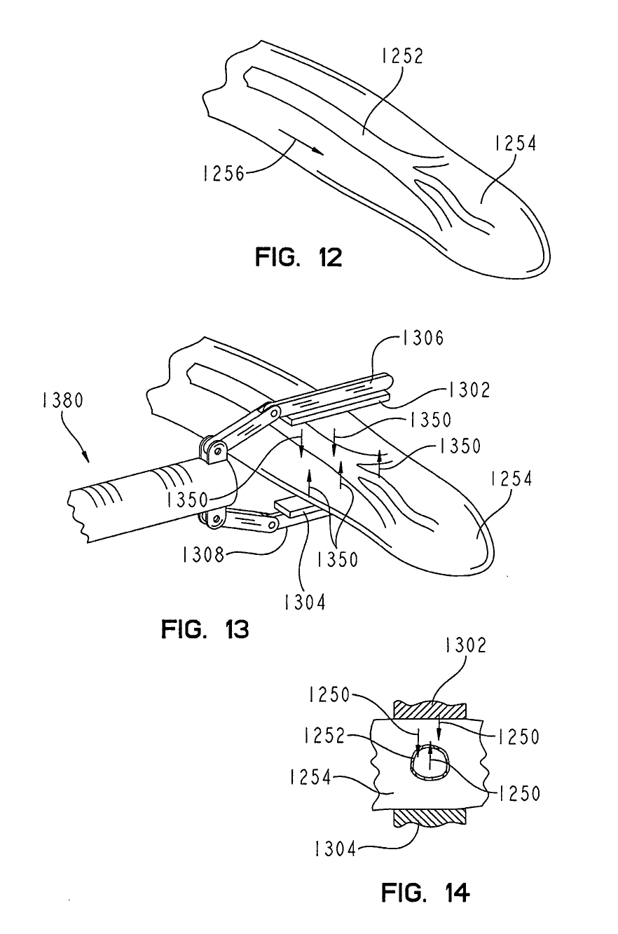

With reference to FIG. 13 there is shown a portion of surgical clamp 1380 which has been introduced to a surgical site and positioned around organ 1254 which was previously described. Clamp 1380 is positioned so that clamp arms 1304 and 1306 are on either side of a portion of organ 1254. As was previously described, an ultrasound transducer 1302 is coupled to clamp arm 1306 and an ultrasound reflector 1304 is coupled to clamp arm 1308. Thus, ultrasound transducer 1302 and ultrasound reflector 1304 are positioned to be on either side of the portion of organ 1254. In this position clamp arms 1306 and 1308 can be moved as was previously described to a relatively closed position so as to exert force on organ 1254 in the locations and directions generally indicated by arrows 1350. Thus, the clamp 1380 can exert force on organ 1254 in the location of ultrasound transducer 1302 and ultrasound reflector 1304 and can cause ultrasound transducer 1302 and ultrasound reflector 1304 to contact organ 1254. Using this positioning capability a variety of diagnostic and therapeutic processes can be performed. Furthermore, stabilization, positioning, compression, and manipulation of organ 1254 can be accomplished. Additionally, the clamping capability can reduce or eliminate the need for additional vascular clamping during a procedure, for example arterial and/or venous clamping upstream from an organ or tissue targeted for therapy.

With reference to FIG. 14 there is shown a partial sectional view of ultrasound transducer 1302 organ 1254 and ultrasound reflector 1304. As illustrated in FIG. 14 ultrasound reflector 1302 and ultrasound reflector 1304 have been positioned to contact organ 1254. In this position ultrasound transducer can operate in a Doppler ultrasound mode to detect blood flow in organ 1254. In Doppler ultrasound mode ultrasound transducer 1302 emits ultrasound which is reflected by red blood cells in vasculature 1252 of organ 1254 and received by transducer 1302. This reflection of Doppler ultrasound is illustrated generally by arrows 1250.

Doppler ultrasound techniques such as the technique illustrated in FIG. 14 can be used to determine whether there is blood flow occurring between transducer 1302 and ultrasound reflector 1304 and the rate of that bloodflow. This determination can be used as the basis for determining whether increased mechanical compression of the clamp is needed to stop or significantly reduce the blood flow prior to the administration of a primary therapy such as administration of high intensity ultrasound, high intensity reflected ultrasound, high intensity unfocused ultrasound or high intensity focused ultrasound via transducer 1302, or administration of an independent primary therapy such as conventional surgical intervention, laser surgical intervention or other surgical intervention to remove part of organ 1254. Doppler ultrasound techniques can also be used during or after administration of such ultrasound therapy techniques to verify the degree of tissue cauterization and to determine whether additional therapy was indicated, for example, a reading of no blood flow would indicate sufficient sealing off of all significant arterial, venous and capillary blood flow after the pressure of a surgical clamp is released. Graphical or numeric output indicating the rate of blood flow can be shown on digital display 248 or monitor 250 of monitor/controller 230 as described above in connection with FIG. 2. Location and volume of blood flow can also be similarly shown or depicted.

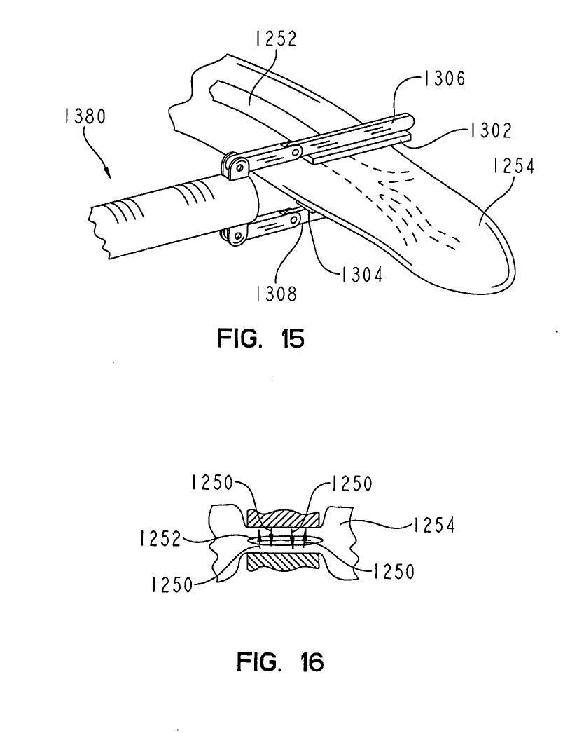

With reference to FIG. 15 there is shown a portion of surgical clamp 1380 which has been introduced to a surgical site, positioned around organ 1254, and adjusted to clamp organ 1254. The clamping is accomplished by clap arms 1304 and 1306 on either side of a portion of organ 1254 being moved to a relatively closed position so as to exert force on organ 1254 as was previously described. In the FIG. 15 the clamping force exerted on organ 1254 is sufficient to substantially interrupt or stop blood flow in vasculature 1252 beyond the clamped portion as is indicated by the dashed portions of vasculature 1252. Using this clamping capability a variety of diagnostic and therapeutic processes can be performed.

With reference to FIG. 16 there is shown an example of therapy and/or diagnostic techniques performed on organ 1254 with vasculature 1252 in due to clamping of organ 1254. As represented generally by arrows 1250, a therapeutic or diagnostic ultrasound can be delivered in this configuration. For example, therapeutic ultrasound in the form of high intensity ultrasound, high intensity reflected ultrasound, high intensity unfocused ultrasound or high intensity focused ultrasound via an ultrasound transducer and/or reflector which are illustrated in cross section above and below organ 1254, and could have characteristics the same as or similar to ultrasound transducers and reflectors that were previously described. Diagnostic Doppler ultrasound may also be administered in this configuration to confirm the interruption of blood flow. It is also contemplated that elastography techniques could be administered in connection with the configuration illustrated in FIG. 16.

Elastography ultrasound techniques can be used to determine the stiffness or compressibility of tissue of organ 1252 which will vary, for example, before and after organ 1252 is subjected to high intensity ultrasound. Elastography techniques involve a comparison of the response of tissue subjected to ultrasound before and after a slight compression, for example, on the order of about 1%-2% compression, or a different percent compression depending upon the tissue involved. Comparison of ultrasound data or images of a tissue subjected to ultrasound from before compression and after compression can be used to determine the degree of compression that the tissue experiences when subjected to a given force. The data collected before and after compression are compared to determine the amount of displacement each small portion of tissue undergoes in response to the compression applied by an ultrasound transducer as illustrated in FIG. 16. The compression can be on the order of about 0.1 to 1 mm, or can vary according to the particular tissue being examined. Furthermore, the compressive force can be determined with a pressure transducer coupled or located adjacent to the ultrasound transducer, or can be determined for a particular clamp and correlated to the indexed control provided by grip locking mechanism 354 described above in connection with FIG. 4. The rate of change of displacement of the tissue as a function of distance from the transducer causing the compression can be used to display a strain image or elastogram or can be presented numerically on monitor 250 or digital display 248 of monitor/controller 230 as described above in connection with FIG. 2.

Elastography techniques can be employed to provide a variety of diagnostic information. For example, tissue stiffness or compressibility will vary according to the degree of tissue cauterization by high intensity ultrasound. Cauterized tissue that has lost its cellular structure has a very different compression ratio from that of normal healthy or viable tissue. Thus elastography techniques can be used to determine the degree of cauterization of a tissue subjected to high intensity ultrasound and to determine whether further application of such therapy is indicated. Similarly, cancerous tissue has a different elasticity coefficient than benign tumor tissue or healthy tissue. Thus, diagnostic elastography ultrasound techniques can be used to locate a tumor or lesion within a tissue or organ for proper positioning of an ultrasound transducer for application of high intensity ultrasound, interruption of blood flow or other therapy.

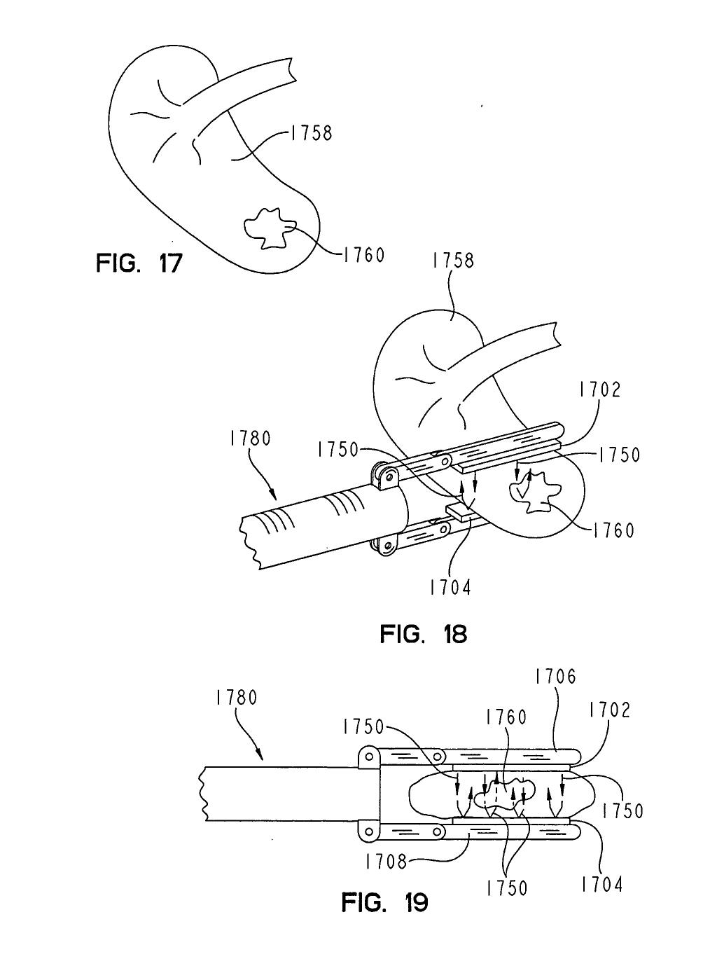

With reference to FIG. 17, there is shown an organ 1758 with a tumor 1760. Organ 1758 is illustrated as a kidney, but could be any organ or tissue including those described above. Tumor 1760 is illustrated generically and could be any type of tumor, lesion or injury.

With reference to FIGS. 18 and 19 there is shown a portion of a surgical clamp 1780 including ultrasound transducer 1702 and an ultrasound reflector 1704. As illustrated in FIG. 18, clamp 1780 can be positioned around and can clamp a portion of organ 1758 as was previously described. In this position ultrasound can be administered to organ 1758 as generally illustrated by arrows 1750. As illustrated, arrows 1750 are high intensity unfocused ultrasound that is emitted form ultrasound transducer 1702 and reflected by ultrasound reflector 1704 to cauterize a region of tissue in organ 1758. It is also contemplated that a variety of other diagnostic and therapeutic ultrasound techniques can be performed on organ 1758 including those described above. With specific reference to FIG. 19 there is shown a side view of the arrangement of FIG. 18. As illustrated in FIG. 19, high intensity ultrasound can be administered to cauterize a plane of tissue defined by the area between transducer 1702 and reflector 1704.

There are a variety of diagnostic and surgical methods according to the present invention. It is contemplated that ultrasonic surgical clamps according to the present invention may be used to deliver adjunctive and primary therapies to a variety of organs or tissues. For example, ultrasonic surgical clamps according to the present invention can be used to grasp, immobilize stabilize, evaluate and cauterize portions of the kidney, liver, blood vessels, and other organs. Such clamps are also contemplated as being useful in performing appendectomies, hysterectomies and removal of other organs and tissues or portions thereof.

In one method according to the present invention an ultrasonic surgical clamp is introduced into a patient via a conventional surgical opening or an endoscopic surgical opening. The clamp is positioned around a tissue or organ and clamps the tissue or organ. The ultrasound transducer is operated in a Doppler diagnostic mode to determine a rate of blood flow in the organ or tissue. Information of the rate of blood flow is displayed on a screen and, if desired the clamp is tightened. These steps are repeated until the rate of blood flow is reduced to a desired level or substantially stopped. Once the desired clamping effect is achieved additional therapy can be administered to the tissue or organ. For example, an ultrasound transducer of the ultrasonic surgical clamp can emit high intensity unfocused ultrasound which is reflected by the reflector of the clamp effective to cauterize tissue intermediate the transducer and the reflector.

In another method according to the present invention an ultrasonic surgical clamp is introduced into a patient via a conventional surgical opening or an endoscopic surgical opening, the clamp is positioned to clamp an organ or tissue, the ultrasound transducer of the clamp is operated to emit high intensity unfocused ultrasound which is reflected by the reflector of the clamp and is effective to cauterize at least part of the tissue or organ intermediate the transducer and the reflector. The transducer is then operated in an elastography mode to measure the elastic properties of the tissue. Based on the result of the elastography the degree of cauterization of tissue can be determined.

While the invention has been illustrated and described in detail in the drawings and foregoing description, the same is to be considered as illustrative and not restrictive in character, it being understood that only the preferred embodiment has been shown and described and that all changes and modifications that come within the spirit of the invention are desired to be protected.

What is claimed is:

1. An apparatus comprising:
a surgical clamp including a first arm and a second arm;
a source of high intensity unfocused ultrasound, the source being coupled to the first arm; and
an ultrasound reflector coupled to the second arm;
wherein at least one of the first arm and the second arm is moveable to position the reflector to reflect non-focused high intensity ultrasound emitted by the source and to clamp an organ or tissue intermediate the source and the reflector.

2. The apparatus of claim 1 further comprising a first linkage connected to the first arm and a second linkage connected to the second arm wherein the first linkage and the second linkage maintain the emitter and the reflector in a substantially parallel relationship upon said movement of at least one of the first arm and the second arm.

3. The apparatus of claim 1 wherein the source comprises a flat ultrasound transducer or an array of hinged ultrasound transducers.

4. The apparatus of claim 1 further comprising;
a handle including operator controls, the handle releasably connectable to the clamp;
an electrical interconnection between the handle and the clamp effective to provide power to the source of high intensity unfocused ultrasound; and
a mechanical interconnection between the handle and the clamp operable to provide mechanical energy to the at least one of the first arm and the second arm which is moveable to clamp an organ or tissue.

5. The apparatus of claim 4 wherein the surgical clamp is a disposable or single use device.

6. The apparatus of claim 1 wherein both the first arm and the second arm are moveable.

7. A system comprising:
a surgical clamp including a multi-mode ultrasound transducer; and
a controller operatively coupled to the transducer to power the transducer;
wherein the controller is operable in a first mode to power the transducer to emit therapeutic ultrasound and is further operable in a second mode to power the transducer to emit diagnostic ultrasound.

8. The system of claim 7 wherein the first mode to power the transducer to emit therapeutic ultrasound powers the transducer to emit unfocused high intensity ultrasound.

9. The system of claim 7 wherein the second mode to power the transducer to emit diagnostic ultrasound powers the transducer to emit Doppler ultrasound.

10. The system of claim 7 wherein the second mode to power the transducer to emit diagnostic ultrasound powers the transducer to emit elastography ultrasound.

11. The system of claim 7 wherein the controller further includes a display device operable to display information based upon the diagnostic ultrasound.

12. The system of claim 7 wherein the controller is operable in a third mode to power the transducer to emit second diagnostic ultrasound.

13. A method comprising:
providing a medical instrument including a clamp and an ultrasound transducer coupled to the clamp;
clamping tissue with the clamp;
subjecting tissue clamped with the clamp to high intensity ultrasound emitted by the transducer; and
determining a characteristic of tissue clamped with the clamp using ultrasound emitted by the transducer.

14. The method of claim 13 wherein the ultrasound emitted by the transducer is high intensity unfocused ultrasound.

15. The method of claim 13 wherein the determining includes one of determining bloodflow using Doppler ultrasound emitted by the transducer and determining a mechanical characteristic of tissue using elastography ultrasound emitted by the transducer.

16. The method of claim 13 wherein the clamping is effective to interrupt bloodflow in the tissue.

17. The method of claim 13 wherein the determining occurs before the subjecting and the determining is effective to provide an indication of a bloodflow in the tissue.

18. The method of claim 13 wherein the subjecting occurs before the determining and the determining is effective to provide an indication of a degree of tissue cauterization.

19. The method of claim 13 further comprising second determining a second characteristic of the clamped tissue using ultrasound emitted by the transducer.

20. The method of claim 19 wherein the determining is effective to provide an indication of bloodflow and the second determining is effective to provide an indication of a degree of tissue cauterization.
